(19) United States
(12) Patent Application Publication
(10) Pub. No.: US 2016/0199131 A1
(43) Pub. Date: Jul. 14, 2016

(54) TEMPERATURE SENSING AND TISSUE ABLATION USING A PLURALITY OF ELECTRODES

(71) Applicant: Advanced Cardiac Therapeutics, Inc., Santa Clara, CA (US)

(72) Inventors: Robert C. Allison, Rancho Palos Verdes, CA (US); John F. McCarthy, Newbury, NH (US)

(73) Assignees: Advanced Cardiac Therapeutics, Inc., Santa Clara, CA (US); Meridian Medical Systems, LLC, Portland, ME (US)

(21) Appl. No.: 15/074,935

(22) Filed: Mar. 18, 2016

Related U.S. Application Data
(63) Continuation of application No. PCT/US2014/056131, filed on Sep. 17, 2014.
(60) Provisional application No. 61/880,425, filed on Sep. 20, 2013.

(51) Int. Cl. A61B 18/18; A61B 18/14; A61B 18/02

(52) U.S. Cl. CPC A61B 18/1815; A61B 18/0206; A61B 18/1492; A61B 18/18; A61B 2018/0016

(57) ABSTRACT

According to some embodiments, an ablation system or other treatment system comprises an elongate body, a first energy delivery member positioned along the distal end of the elongate body, and at least a second energy delivery member positioned at a location proximal to the first energy delivery member, the first energy delivery member and the second energy delivery member being configured to deliver energy sufficient to at least partially ablate tissue. In some embodiments, each of the first and second energy delivery members comprises an antenna configured to receive a microwave signal corresponding to a temperature of the tissue at a location adjacent the antenna. The system further comprises at least one radiometer configured to process the microwave signals received from the antennas of the energy delivery members and configured to produce an output signal representative of tissue temperatures at depth adjacent the first and second energy delivery members.

### INCORPORATION BY REFERENCE TO PRIORITY APPLICATIONS

The present application is a continuation of PCT Patent Application No. PCT/US2014/056131, filed Sep. 17, 2014, which claims priority benefit of U.S. Provisional Patent Application No. 61/880,425, filed on Sep. 20, 2013. Both of the foregoing priority applications are hereby incorporated by reference in their entirety.

### FIELD

The present application relates to ablation systems, and more specifically to ablation systems configured for radiometric temperature sensing using a plurality of electrodes or other energy delivery members.

### BACKGROUND

Tissue ablation may be used to treat a variety of clinical disorders. For example, tissue ablation may be used to treat cardiac arrhythmias by destroying (e.g., at least partially or completely ablating, interrupting, inhibiting, terminating conduction of, otherwise affecting, etc.) aberrant pathways that would otherwise conduct abnormal electrical signals to the heart muscle. Several ablation techniques have been developed, including cryoablation, microwave ablation, radiofrequency (RF) ablation, and high frequency ultrasound ablation. For cardiac applications, such techniques are typically performed by a clinician who introduces a catheter having an ablative tip to the endocardium via the venous vasculature, positions the tip adjacent to what the clinician believes to be an appropriate region of the endocardium based on tactile feedback, mapping electrocardiogram (ECG) signals, anatomy, and/or fluoroscopic imaging, actuates flow of an irrigant to cool the surface of the selected region, and then actuates the tip for a period of time and at a power believed sufficient to destroy tissue in the selected region.

### SUMMARY

According to some embodiments, an ablation system or other treatment system comprises an elongate body (e.g., a catheter, other medical instrument, etc.) having a proximal end and a distal end, a first energy delivery member positioned along the distal end of the elongate body, and at least a second energy delivery member positioned at a location proximal to the first energy delivery member, the first energy delivery member and the second energy delivery member being configured to deliver energy sufficient to at least partially ablate tissue. In some embodiments, each of the first and second energy delivery members comprises an antenna configured to receive a microwave signal corresponding to a temperature of the tissue at a location adjacent the antenna. The system further comprises at least one radiometer configured to process the microwave signals received from the antennas of the first and second energy delivery members, the at least one radiometer being configured to produce an output signal representative of tissue temperatures at depth adjacent the first and second energy delivery members. In some embodiments, the system additionally comprises an energy delivery module (e.g., a generator) and at least one conductor coupling the first and second energy delivery members to the energy delivery module.

According to some embodiments, each of the first and second energy delivery members comprises a radiofrequency (RF) electrode. In some embodiments, the RF electrode comprises a ring electrode. In other embodiments, the electrode comprises a non-ring electrode. In one embodiment, each of the first and second energy delivery members comprises a microwave emitter, an ultrasound transducer, an optical emitter, a cryoablation member and/or any other energy delivery device or feature. In some embodiments, the system comprises two or more (e.g., 3, 4, 5, 6, 7, 8, 9, 10 energy delivery members, more than 10 energy delivery members, etc.).

According to some embodiments, each of the antennas of the first and second energy delivery members comprises a helical antenna. In one embodiment, each of the first and second energy delivery members extends circumferentially around the elongate body. In some embodiments, each of the first and second energy delivery members does not extend circumferentially around the elongate body. In one embodiment, one energy delivery member is radially offset from at least one other energy delivery member.

According to some embodiments, the first and second energy delivery members are positioned on an expandable member. In one embodiment, the expandable member comprises an inflatable balloon (e.g., compliant or non-compliant balloon). In some embodiments, the expandable member comprises an expandable cage, basket, scaffold or other expandable mechanical structure (e.g., comprising a plurality of struts, fingers, prongs and/or the like). In some embodiments, at least some of the energy delivery members are positioned along the plurality of struts or other expandable structure.

According to some embodiments, the elongate body comprises at least one irrigation passage. In one embodiment, the irrigation passage extends at least partially through the catheter (e.g., to or near the distal end of the catheter). In some embodiments, the irrigation passage extends to at least one of the energy delivery members. In some embodiments, the irrigation passage is part of an open irrigation system, wherein fluid delivered through the irrigation passage exits the elongate body near at least one of the energy delivery members. In some embodiments, the irrigation passage is part of a closed irrigation system, wherein fluid is circulated through an interior of the elongate body to facilitate heat transfer.

According to some embodiments, the system additionally comprises an electrophysiology recorder. In some embodiments, the energy delivery module comprises a generator (e.g., RF generator, another type of power or energy provider, etc.).

According to some embodiments, the energy delivered by the energy delivery module is automatically regulated based on a control scheme. In one embodiment, the energy delivered by the energy delivery module is automatically regulated to maintain a desired temperature setpoint or range along the targeted tissue. In some embodiments, the energy delivered by the energy delivery module is automatically regulated to create a desired heating profile along the targeted tissue. In one embodiment, the heating profile along the energy delivery members is generally constant or even. In some embodiments, the heating profile along the first and second energy delivery members varies along the targeted tissue (e.g., the heating profile is linear or non-linear).

According to some embodiments, the energy delivered by the delivery module is manually regulated by a physician or other user. In one embodiment, the physician or other user regulates the power to one or more of the energy delivery members by viewing real-time temperature data obtained using the radiometry. In some embodiments, the temperature data is provided to the physician or user via a display. In some embodiments, the physician or other user can regulate which energy delivery member is energized and can regulate at least one parameter related to the operation of the energy delivery members.

According to some embodiments, the system further includes at least one switch configured to receive the microwave signals from the antennas of the first and second energy delivery members and to multiplex said microwave signals. In some embodiments, each of the first and second energy delivery members comprises a diplexer to permit said energy delivery member to deliver energy to tissue when energized and to receive microwave signals emitted by the tissue.

According to some embodiments, a method of determining a temperature and facilitating ablation of tissue of a subject comprises determining a temperature of a tissue of a subject at a depth relative to the tissue's surface along at least two longitudinal locations of a catheter, each of said locations corresponding to a location of one of a plurality of energy delivery members positioned on the catheter, wherein determining the temperature comprises receiving microwave energy emitted by tissue at each of the plurality of energy delivery members using an antenna of each of the energy delivery members and providing a corresponding microwave signal from each antenna to a radiometer. In some embodiments, the method further comprises delivering energy to the tissue of the subject by activating the plurality of energy delivery members.

According to some embodiments, each of the energy delivery members comprises a radiofrequency (RF) electrode. In some embodiments, the RF electrode comprises a ring electrode. In one embodiment, each of the energy delivery members comprises a microwave emitter, an ultrasound transducer, an optical emitter, a cryoablation member and/or any other energy delivery device or feature. In some embodiments, the system comprises two or more (e.g., 3, 4, 5, 6, 7, 8, 9, 10 energy delivery members, more than 10 energy delivery members, etc.).

According to some embodiments, each of the antennas of the energy delivery members comprises a helical antenna. In one embodiment, each of the energy delivery members extends circumferentially around the elongate body. In some embodiments, each of the energy delivery members does not extend circumferentially around the elongate body. In one embodiment, one energy delivery member is radially offset from at least one other energy delivery member.

According to some embodiments, the energy delivery members are positioned on an expandable member. In one embodiment, the expandable member comprises an inflatable balloon (e.g., compliant or non-compliant balloon). In some embodiments, the expandable member comprises an expandable cage, basket, scaffold or other expandable mechanical structure (e.g., comprising a plurality of struts, fingers, prongs and/or the like). In some embodiments, at least some of the energy delivery members are positioned along the plurality of struts or other expandable structure.

According to some embodiments, the elongate body comprises at least one irrigation passage. In one embodiment, the irrigation passage extends at least partially through the catheter (e.g., to or near the distal end of the catheter). In some embodiments, the irrigation passage extends to at least one of the energy delivery members. In some embodiments, the irrigation passage is part of an open irrigation system, wherein fluid delivered through the irrigation passage exits the elongate body near at least one of the energy delivery members. In some embodiments, the irrigation passage is part of a closed irrigation system, wherein fluid is circulated through an interior of the elongate body to facilitate heat transfer.

In one embodiment, each transducer (e.g., electrode, other energy delivery member, etc.) of an ablation catheter is placed at a location and performs both an ablation procedure and temperature sensing. For example, each transducer may comprise a RF electrode, a microwave antenna (e.g., at 4 GHz), a device associated with cryoablation, or any other device capable of performing the ablation procedure at the location. Specifically, each transducer has the ability to deliver a signal for tissue heating (e.g., ablation) at the location, and also the ability to provide a microwave signal, associated with the temperature of the tissue at the location, to a radiometer. Further, a switch may be utilized to multiplex the microwave signals received from the plurality of transducers, wherein the switch may then provide the multiplexed signal to the radiometer. The radiometer may compare an internal reference temperature with temperature information in the multiplexed signal. The comparison may produce an output signal at the radiometer that may then be sampled by a controller to obtain the temperatures at the plurality of locations where the transducers are positioned and performing the ablation procedures. The temperatures may be provided to a physician, for example, in a variety of different forms. Advantageously, the physician may utilize the provided temperatures to increase the precision of cardiac ablation at the plurality of locations associated with the plurality of transducers. Specifically, the physician may variably control the power (e.g., RF signal) to each transducer utilizing the provided temperatures.

### BRIEF DESCRIPTION OF THE DRAWINGS

These and other features, aspects and advantages of the present application are described with reference to drawings of certain embodiments, which are intended to illustrate, but not to limit, the concepts disclosed herein. The attached drawings are provided for the purpose of illustrating concepts of at least some of the embodiments disclosed herein and may not be to scale.

FIG. 1 depicts a view of an example ablation catheter having a plurality of RF electrodes that are positioned within a human body during an ablation procedure;

FIG. 2 depicts a block diagram of an example ablation catheter having the plurality of RF electrodes that perform the ablation procedures and temperature sensing;

FIG. 3 depicts an example display that outputs the temperatures at the locations where the plurality of RF electrodes are positioned and performing the ablation procedure;

FIG. 4 is an example procedure for temperature sensing of an ablation catheter having the plurality of RF electrodes;

FIG. 5 illustrates one embodiment of a RF electrode configured to be positioned along a catheter;

FIG. 6 illustrates a cross-sectional view of a RF electrode configured to be positioned along a catheter;

FIG. 7 illustrates one embodiment of a treatment system comprising a catheter with a plurality of electrodes according to one embodiment; and

FIG. 8 schematically illustrates one embodiment of a treatment system comprising a catheter with a plurality of electrodes arranged in a radial orientation according to one embodiment.

### DETAILED DESCRIPTION

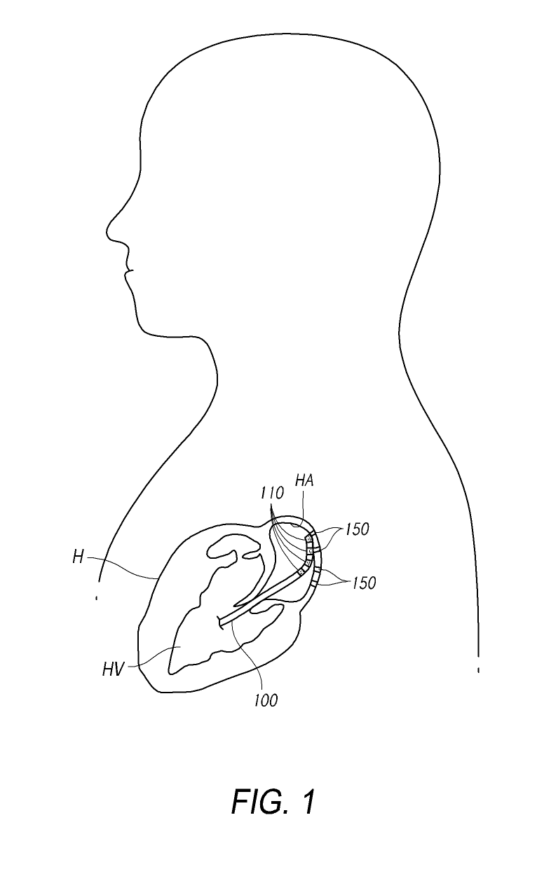

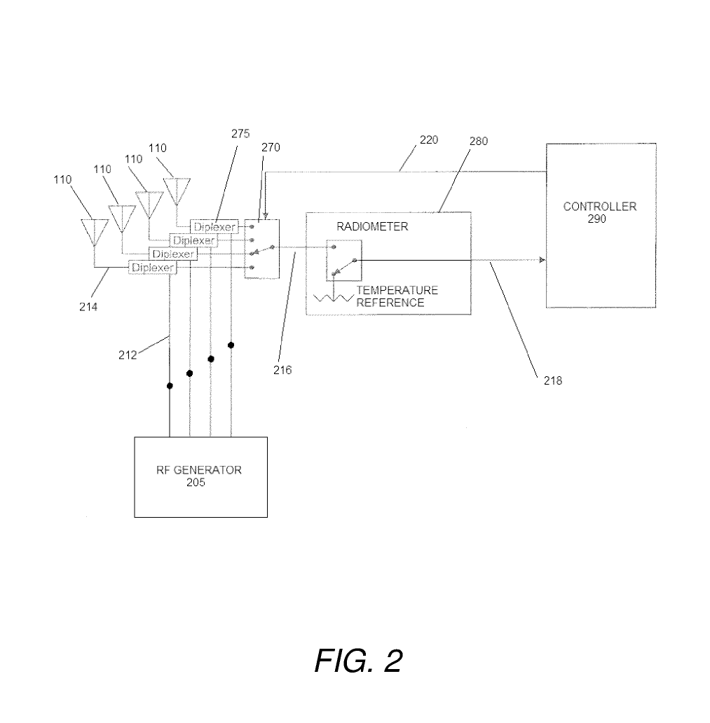

FIG. 1 depicts a view of an example ablation catheter having a plurality of RF electrodes that are positioned within a human body during an ablation procedure. Specifically, FIG. 1 shows a head and torso of a patient having a heart H with a left ventricle HV and a left atrium HA. In some embodiments, during a cardiac ablation procedure, an ablation catheter 100 may be threaded or otherwise advanced into the left atrium HA via the left ventricle HV so that a plurality of RF electrodes 110 contact a posterior wall of the left atrium, as shown in FIG. 1, for example. Specifically, in some embodiments, each RF electrode 110 is provided an RF signal to heat the tissue at locations 150 to perform the ablation or other heat treatment procedure. In addition, each electrode 110 can be advantageously configured to receive microwave energy (e.g., via an antenna) and provide a microwave signal to the radiometer 280 (FIG. 2), as described herein, for temperature sensing. The use of radiometry can allow for the accurate temperature measurement of targeted tissue at a depth, and thus, can facilitate for a more efficacious and safe treatment procedure. In some embodiments, each RF electrode is configured to operate in a range from 100's KHz to several GHz (e.g., between 100 KHz and 10 GHz), as known by those skilled in the art. For example, with respect to sensing, each electrode can be configured to operate between 100 MHz and 20 GHz (e.g., 100-200, 200-300, 300-400, 400-500, 500-600, 600-700, 700-800, 800-900 MHz, 900 MHz-1 GHz, 1-2, 2-3, 3-4, 4-5, 5-6, 6-7, 7-8, 8-9, 9-10, 10-15, 15-20 GHz, frequencies between the foregoing ranges, etc.). In some embodiments, the electrodes are configured to operate in a microwave range of about 500 MHz to 7 GHz. In addition, with respect to ablation, each electrode can be configured to operate between 100 KHz and 10 MHz (e.g., 100-200, 200-300, 300-400, 400-500, 500-600, 600-700, 700-800, 800-900 KHz, 900 KHz-1 MHz, 1-2, 2-3, 3-4, 4-5, 5-6, 6-7, 7-8, 8-9, 9-10 MHz, frequencies between the foregoing ranges, etc.).

The depiction of four RF electrodes 110 in FIG. 1 is only a single embodiment. Thus, any number of RF electrodes 110 may be used in other embodiments. For example, in some embodiments, a multi-electrode system can comprise fewer than four electrodes (e.g., 2 or 3) or more than four electrodes (e.g. 5, 6, 7, 8, 9, 10 electrodes, more than 10 electrodes, etc.), as desired or required. Further, although the RF electrodes 110, as depicted in FIG. 1, are configured to make contact with the posterior of the heart, the RF electrodes may make contact with any area where an ablation procedure may occur (e.g., arteries, veins or other vessels, other organs, other tissue, etc.).

An ablation or other thermal-based treatment system in accordance with the various embodiments disclosed herein can include two or more electrodes positioned on or along a medical instrument (e.g., catheter, other elongate member, etc.). The medical instrument can be sized, shaped and/or otherwise configured to be passed intraluminally (e.g., intravascularly) through a subject being treated. In various embodiments, the medical instrument comprises a catheter, a shaft, a wire, and/or other elongate instrument.

In some embodiments, the medical instrument (e.g., catheter) is operatively coupled to one or more devices or components. For example, the medical instrument can be coupled to an energy delivery module. According to some arrangements, the energy delivery module includes an energy generation device that is configured to selectively energize and/or otherwise activate the energy delivery members (e.g., radiofrequency electrodes) located along the catheter. In some embodiments, for instance, the energy generation device comprises a radiofrequency generator, another type of electrical energy source or generator, a cryogenic fluid source and/or the like. Accordingly, as used herein, energy

The energy delivery module can include one or more input/output devices or components, such as, for example, a touchscreen device, a screen or other display, a controller (e.g., button, knob, switch, dial, etc.), keypad, mouse, joystick, trackpad, or other input device and/or the like. Such devices can permit a physician or other user to enter information into and/or receive information from the system. In some embodiments, the output device can include a touchscreen or other display that provides tissue temperature information, contact information, other measurement information and/or other data or indicators that can be useful for regulating a particular treatment procedure.

According to some embodiments, the energy delivery module includes a processor (e.g., a processing or control unit) that is configured to regulate one or more aspects of the treatment system. The module can also comprise a memory unit or other storage device (e.g., computer readable medium) that can be used to store operational parameters and/or other data related to the operation of the system. In some embodiments, the processor is configured to automatically regulate the delivery of energy from the energy generation device to the RF electrodes or other energy delivery members based on one or more operational schemes. For example, as discussed in greater detail in U.S. patent application Ser. No. 14/285,337, filed on May 22, 2014, the entirety of which is hereby incorporated by reference herein, energy provided to the electrodes (and thus, the amount of heat transferred to or from the targeted tissue) can be regulated based on, among other things, the detected temperature of the tissue being treated along one, some or all of the electrodes.

According to some embodiments, the energy delivery system can include one or more temperature detection devices, such as, for example, reference temperature devices (e.g., thermocouples, thermistors, etc.), radiometers and/or the like. Additional details regarding such temperature detection devices are provided in U.S. patent application Ser. No. 14/285,337, filed on May 22, 2014, the entirety of which is hereby incorporated by reference herein.

The energy delivery system can comprise (or can be configured to be placed in fluid communication with) an irrigation fluid system. In some embodiments, such a fluid system is at least partially separate from the energy delivery module and/or other components of the system. However, in other embodiments, the irrigation fluid system is incorporated, at least partially, into the energy delivery module. The irrigation fluid system can include one or more pumps or other fluid transfer devices that are configured to selectively move fluid through one or more lumens or other passages of the catheter. Such fluid can be used to selectively cool (e.g., transfer heat away from) the energy delivery members (e.g., RF electrodes) and/or the surrounding tissue of the subject during use.

FIG. 2 schematically illustrates one embodiment of a treatment system comprising a catheter having a plurality of RF electrodes. Such electrodes can be configured to provide heat to targeted tissue when energized and to perform temperature sensing. For example, as discussed in greater detail herein, such electrodes can comprise a microwave antenna or similar feature or component that is configured to receive microwave energy.

With continued reference to FIG. 2, the various electrodes or other energy delivery members 110 included in the system can be selectively activated by (e.g., receive RF signals from) an energy delivery module (e.g., a RF generator, other generator or device, thermal device, etc.) 205 over one or more communication links 212. As noted herein, such a system can be used to perform an ablation or other heat treatment procedure (e.g., to heat or cool targeted tissue). In some embodiments, each electrode or other energy delivery member 110 receives a signal from the generator 205 to perform the procedure. Further, each electrode 110 may provide a microwave signal (e.g., that includes temperature information) over one or more communication links 214. In some embodiments, such microwave signals are provided to a switch 270 or other regulation device for temperature sensing during a procedure. In some arrangements, microwave signals at the location where each electrode is positioned are not generated by the ablation catheter. Instead, such microwave signals are naturally-occurring electromagnetic radiation emitted by the tissue that is proportional to the absolute temperature of the tissue. In some embodiments, each electrode is configured to collect or otherwise receive such microwave signals emitted by adjacent tissue of the subject. The switch 270 can multiplex the plurality of microwave signals into a multiplexed signal. The switch 270 may be a microwave switch or any other type of switch or device, as desired or required.

In some embodiments, when receiving microwave signals from the plurality of electrodes 110, the switch 270 can receive one or more logical instructions from a controller 290 over one or more communication links 220. Specifically, one or more logical instructions regulated by the controller can be used to inform the switch 270 as to which electrode is to be “selected” (e.g., select a path to the selected RF electrode) so that the switch 270 may receive the microwave signal from the 10 selected electrode or other energy deliver member. Since each electrode 110 is configured to both deliver energy to adjacent tissue during a treatment procedure and sense temperature of the adjacent tissue (e.g., sending the microwave signal to the switch 270) at two diverse frequencies, a diplexer 275 or similar device can be utilized by each electrode 110 to isolate the two diverse frequencies. Additional details regarding diplexers, electrodes configured to both deliver energy to tissue and receive microwave signals and/or other devices or components that may be included in a radiometry-enabled treatment system are provided in U.S. patent application Ser. No. 14/285,337, filed on May 22, 2014, the entirety of which is incorporated by reference herein.

According to some embodiments, the multiplexed signal is then provided from the switch 270 to one or more radiometers 280 over a communication link 216, for example. In some embodiments, the radiometer 280 operates at a center frequency, for example, 4 GHz. However, in other embodiments, the frequency at which the radiometer 280 operates can be greater than 4 GHz (e.g., 4-5, 5-6, 6-7, 7-8, 8-9, 9-10, 10-20, 20-30 GHz, frequencies between the foregoing ranges, greater than 30 GHz, etc.) or less than 4 GHz (e.g., 0.1-0.2, 0.2-0.3, 0.3-0.4, 0.4-0.5, 0.5-1, 1-2, 2-3, 3-4 GHz, frequencies between the foregoing ranges, less than 1 GHz, etc.), as desired or required.

In some embodiments, the radiometer 280 compares an internal reference temperature with the temperature information in the multiplexed signal received from switch 270. Specifically, the radiometer 280 may determine the difference between the internal reference temperature and the temperature information, associated with each electrode or other energy delivery member in the multiplexed signal, to produce an output signal at the radiometer 280.

In some embodiments, the output signal can be sampled by a controller 290, over one or more communication links 218, for example, to obtain the temperatures at the different locations where the electrodes or other energy delivery members 110 are positioned (e.g., for performing an ablation or other treatment procedure). Specifically, the controller 290 can sample the output signal at the radiometer 280 at a sample rate (e.g., many samples per second, such as, for example, 0-10, 10-20, 20-30, 30-40, 40-50, 50-100, 100-1,000, 1,000-10,000, 10,000-100,000 samples per second, values between the foregoing ranges, more than 100,000 sampled per second, etc.) that is slower than the sample rate that the microwave signals are being provided from the electrodes 110 to the switch 270. In some embodiments, the controller 290 is configured to sample the output signal at the radiometer 280 at a sample rate that is at least two times faster than the rate of change of the information carried by the microwave signals provided from the electrodes 110 to the switch 270.

In some embodiments, as schematically illustrated in FIG. 2, the switch 270 is separate and distinct from the radiometer 280. In alternative embodiments, however, the switch 270 is integrated, at least partially, with the radiometer 280. Further, any number of electrodes, switches, communication links, RF generators, radiometers, controllers and/or other components or devices can be positioned on and/or coupled (e.g., physically, operatively, etc.) to the ablation catheter. Thus, the figures illustrated herein simply depict certain non-limiting embodiments for simplicity and clarity.

In some embodiments, microwave signals received from all the electrodes located along a catheter of the system are directed to a single radiometer. However, in other embodiments, two or more radiometers can be used in a particular system. For example, in such a configuration, one or more of the microwave antennas are configured to transmit their signals to a first radiometer, while one or more other microwave antennas are configured to transmit their signals to a second radiometer. Regardless of how the microwave antennas, radiometer chips and/or other components of the system are configured, however, the use of radiometry can advantageously facilitate the accurate measurement of targeted tissue at depth along one, some or all of the electrodes. Accordingly, in some embodiments, the ability to accurately control the operation of the energy delivery to targeted tissue (e.g., to create a desired heating profile, to maintain the tissue temperature with a setpoint or range, etc.) can be more reliably and accurately achieved.

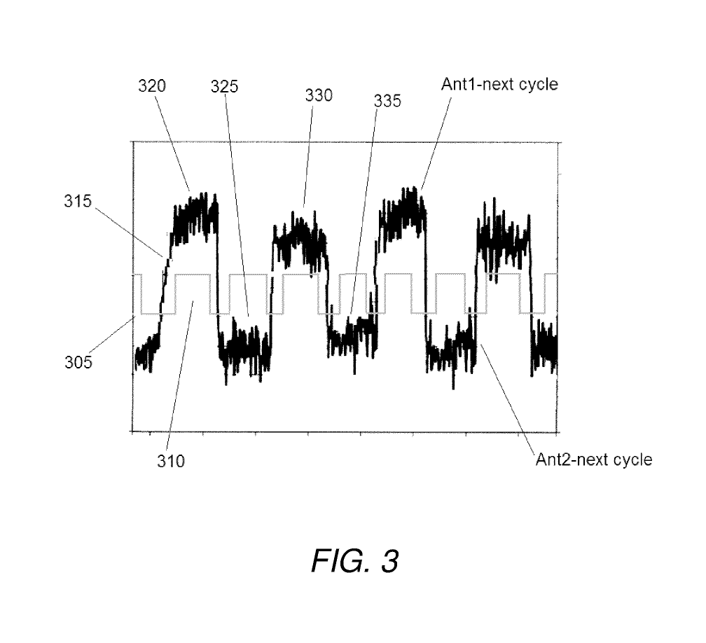

FIG. 3 depicts one embodiment of a display that outputs the temperatures at the locations where the plurality of electrodes 110 or other energy delivery members are positioned and performing the ablation or other treatment procedure. As shown, for example, a clock signal 305 may represent the sampling rate at which the controller 290 samples the output signal at the radiometer, wherein the clock signal may include one or more pulses 310. For example, in some embodiments, the controller 290 averages samples during each 40 ms pulse. In other embodiments, the pulse can be greater than 40 ms (e.g., 40-45, 45-50, 50-60, 60-70, 70-80, 80-90, 90-100, 100-200, 200-300, 300-400, 400-500 ms, values between the foregoing ranges, greater than 500 ms, etc.) or less than 40 ms (e.g., 0-5, 5-10, 10-15, 15-20, 20-25, 25-30, 30-35, 35-40 ms, values between the foregoing ranges, etc.), as desired or required. In some embodiments, the x-axis in FIG. 3 corresponds to time, and the clock signal is represented by 305. Further, in some embodiments, the y-axis corresponds to a temperature, where a higher temperature value would be located at a higher y-axis value.

Further, in some embodiments, a signal 315 (e.g., waveform) may have portions 320, 325, 330, 335 that correspond to each of the four electrodes 110, as described herein, for example, with reference to FIGS. 1 and 2. Specifically, the portion 320 that corresponds to a first electrode of the plurality of electrodes 110 shows that the location where the first electrode 110 is positioned (e.g., within the patient's body) is “hot” in temperature (e.g., having a higher temperature relative to a baseline or threshold) since the portion 320 is located at a relatively high y-axis value. Further, in the illustrated embodiment, the portion 325 that corresponds to a second electrode of the plurality of electrodes 110 indicates that the location where the second electrode 110 is positioned is “cold” in temperature (e.g., having a lower temperature relative to a baseline or threshold) since the portion 325 is located at a relatively low y-axis value. The portion 330 that corresponds to a third electrode of the plurality of electrodes 110 shows that the location where the third electrode 110 is positioned is “hot” in temperature since the portion 330 is located at a relatively high y-axis value. Finally, the portion 335 that corresponds to a fourth electrode of the plurality of electrodes 110 shows that the location where the fourth electrode 110 is positioned is “cold” in temperature since the portion 335 is located at a relatively low y-axis value.

According to some embodiments, the power provided to one or more of the electrodes or other energy delivery members can be adjusted to maintain a desired temperature along the catheter (e.g., at the various locations of the electrodes or other energy delivery members). In some embodiments, the electrodes or other energy delivery members can be regulated in order to create a desired or required heating (or cooling) profile along the targeted tissue (e.g., atrial or ventricular tissue, other cardiac tissue, pulmonary vein, other vessels or lumens, other tissue, etc.). For example, a physician or other practitioner may utilize the output as depicted in FIG. 3 to view the temperatures at the locations 150 to increase the precision of a treatment procedure (e.g., ablation, other heating or cooling procedure, etc.) at the locations 150 associated with the plurality of electrodes 110. For example, the physician may increase the strength of the signal at the locations where the second and fourth electrodes are positioned, since the temperature at the locations is deemed “cold.” Further, the physician may decrease the strength of the signal at the locations where the first and third electrodes are positioned, since the temperature at the locations is deemed “hot.” In any case, depending on the particular control scheme and/or other factors or circumstances, a physician may variably control the power (e.g., RF signal) supplied to the electrode to increase the precision of cardiac ablation utilizing the temperatures information provided by the electrodes during the ablation procedures. FIG. 3 shows one embodiment of a waveform depicting the temperature at the locations 150 of the electrodes 110. However, other techniques may be utilized to inform the physician of the temperatures at locations 150 of the electrodes 110. For example, a digital temperature read out, or any form of graphical presentation associated with one or more electrodes, may be provided to the physician (e.g., via touchscreen, other display or output device, etc.).

As noted above, for any of the embodiments disclosed herein, the control of the various electrodes or other energy delivery members (e.g., RF electrodes, microwave emitters, ultrasound transducers, cryoablation members, etc.) can be automatically or semi-automatically regulated based on one or more control schemes (e.g., closed-loop control, other feedback loop, etc.). As used herein, energy delivery member is a broad term and includes, without limitation, a device that is configured to deliver electrical energy, acoustic or other mechanical energy, thermal energy (e.g., to heat or cool and/or otherwise impact targeted tissue) and/or the like. In some embodiments, the electrodes can be regulated to create a particular temperature profile along the targeted tissue. In other embodiments, a control scheme ensures that the electrode temperature and/or the tissue temperature does not exceed a particular threshold value (e.g., high temperature threshold, low temperature threshold, etc.) during or as a result of a treatment procedure. In some embodiments, a control scheme is used to ensure that the temperature of the targeted tissue remains within a desired or required temperature range.

In some embodiments, the output of the radiometer at a particular electrode is used to control the power supplied by the RF generator to that electrode in order to maintain the temperature of the tissue adjacent that particular antenna/RF electrode at a desired temperature setpoint or range. In some embodiments, such a closed-loop scheme is implemented at each of the electrodes. Thus, the antennas/RF electrodes can be multiplexed to permit the system to create a lesion with a desired temperature profile. As noted herein, in some embodiments, such a temperature profile is generally uniform along the section of the catheter that comprises the electrodes. However, in other arrangements, the profile can be non-uniform (e.g., sloped wherein one or more portions or sections (e.g., distal, proximal, middle, etc.) of the catheter are warmer or cooler than other portions or sections), as desired or required. The desired temperature profile can be linear or non-linear (e.g., curved, sinusoidal, logarithmic, irregular, etc.).

According to some embodiments, the various electrodes or other energy delivery members included in a particular system are electrically coupled to a single energy delivery module (e.g., a RF generator). However, in other embodiments, two or more separate energy delivery modules can be used to selectively activate the electrodes or other energy delivery members of the catheter. In some embodiments, the RF generator or other energy delivery module is connected or otherwise coupled to an array of switches that can be used to regulate power to each of the electrodes. Further, the system can also include a controller (e.g., a microcontroller, other controller, etc.) that is connected or otherwise operatively coupled (e.g., directly or indirectly) to each switch. Such a controller can be used to selectively control the switch (e.g., between on and off positions) in order to provide power to the various electrodes.

According to some embodiments, one or more power transistors, diodes, resistors, control buses, multiplexers, amplifiers, converters, wires or other conductors and/or other electrical components can be used to regulate the activation, deactivation and/or modulation of each of the electrodes included within a system. Regardless of the exact design and configuration of the system, a control scheme can be created to regulate the activation of the various electrodes during a treatment procedure in order to achieve a desired result. For example, in some embodiments, the system is regulated so as to maintain the temperature of the targeted tissue at a particular setpoint or within a particular range. In some embodiments, the system can be regulated so as to create a desired heating profile along the targeted tissue. For example, the electrodes of the system can be operated to maintain the portion or area of the subject's tissue that is targeted at a generally constant temperature. In other arrangements, however, the system can be configured to create sections or portions of the targeted tissue hotter or cooler than others, as desired or required.

In some embodiments, localized radiometric temperature measurements of tissue received from each of the electrodes can be provided to a processor or other controller to control the energy provided to each of the electrodes. For example, the manner in which the electrodes are activated (e.g., turned on or off, modulated between a lower or higher power level, pulsed, etc.) can be based on a selected control scheme. Such a control scheme can incorporate closed-loop control to maintain the temperature of the targeted tissue at a desired setpoint or range. In some embodiments, a user (e.g., physician, other practitioner, etc.) can select a target temperature setpoint or range for a particular procedure (e.g., a tissue ablation procedure), and the system can be automatically regulated (e.g., via selective activation, deactivation and/or power modulation of the various electrodes) to achieve the desired result. In other embodiments, however, as discussed herein, a physician or other practitioner can manually adjust the operational parameter of one or more of the electrodes during a procedure. For example, in such arrangements, the physician can manually adjust the operation of the electrodes based on real-time tissue temperature measurements that are obtained along or near one or more of the electrode locations and displayed to the physician.

In some embodiments, a controller or processor is configured to adjust the feedback signals from a generator to the various electrodes based on the radiometric temperature measurements. Thus, the amplitude of the voltage (or current) generated by the energy delivery module (e.g., RF generator) can be adjusted based on a particular control scheme.

The exact manner in which the system controls the delivery of energy at each of the electrodes can be based on any one of a number of models. In some embodiments, one or more operational parameters of the system's electrodes can be regulated based on a collective duty cycle model. For example, the generator can be configured to deliver power sequentially to each of the electrodes (e.g., in a pulsed manner). The power delivered to each electrode can be constant or can be adjusted in order to more accurately control the resulting heating profile of the targeted tissue (e.g., based on the radiometric temperature measurements that are obtained at or along each of the electrodes). In some embodiments, the processor can sequence successive power pulses to adjacent electrodes so that the end of the duty cycle for a preceding pulse overlaps, at least partially, with the beginning of the duty cycle for the next pulse. Such an overlap can help ensure that power is applied to the various electrodes continuously, with no periods of interruption caused by open circuits during pulse switching between successive electrodes.

In other embodiments, the system's processor is configured to make individual adjustments to the power (e.g., the amplitude of the RF voltage) provided to each electrode without pulsing. Thus, in such a configuration, the processor is able to selectively operate each of the electrodes based on the tissue temperature feedback that is received by the system. The system can be configured to adjust the operational parameters of one or more of the electrodes without having to sequence through all of the electrodes. In such embodiments, power supplied by the generator can be non-continuous (e.g., can include interruptions in power delivery).

As discussed herein, according to some embodiments, one or more operational parameters (e.g., activation/deactivation, modulation at various power levels, duration of activation, etc.) of the various electrodes can be based on an automatic control scheme. For example, the physician or other practitioner can be prompted (e.g., prior to beginning a treatment procedure) to enter a desired setpoint temperature or range. In some embodiments, such a setpoint temperature or range is representative of the peak temperature of the targeted tissue that is desired to be achieved as a result of the procedure. For example, in some embodiments, such a setpoint or range helps ensure that a desired tissue lesion, ablation and/or other clinical result is adequately performed. Accordingly, in such embodiments, regardless of the exact manner in which the electrodes are controlled and operated (e.g., randomly or sequentially activated or pulsed), a processor of the system can be configured to compare the target setpoint or range with the tissue temperature at depth obtained along each of the electrodes and make corresponding operational changes to one or more of the electrodes to attain the goal.

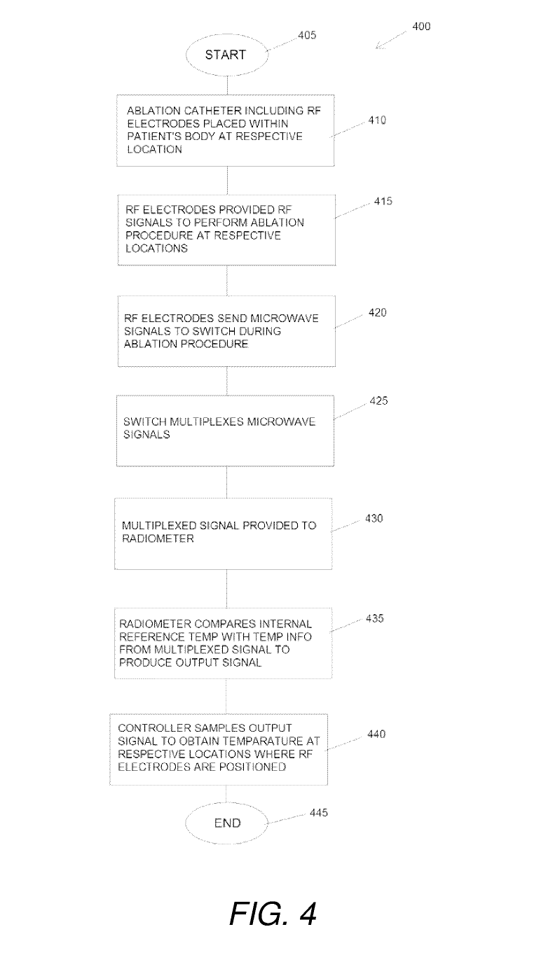

FIG. 4 illustrates a flowchart 400 of one embodiment of a procedure for temperature sensing of an ablation catheter having a plurality of RF electrodes. As shown, the procedure 400 can start at step 405 and continue to step 410 where an ablation catheter having a plurality of RF electrodes may be placed within a patient's body at respective locations. For example, the RF electrodes may make contact with a wall or other tissue of the heart, where the ablation procedure is set to occur. However, as noted herein, any other type of tissue can be targeted with the treatment systems described herein, such as, for example, vessels or other bodily lumens, other organs, nerve tissue and/or the like.

With continued reference the embodiment illustrated in FIG. 4, at step 415, each of the plurality of RF electrodes can energized or activated (e.g., provided with a RF signal) to perform the ablation or other treatment procedure at the respective locations where each RF electrode is positioned. For example, a RF generator may provide each electrode an RF signal to perform the procedure at the respective locations. At step 420, in some embodiments, while the procedure is occurring at the different locations, each RF electrode may provide a microwave signal to a switch. As noted herein, each electrode can comprise a microwave antenna that is configured to receive corresponding signals (e.g., microwave signals emitted by tissue adjacent to the antenna and electrode).

According to some embodiments, at step 425, the switch may multiplex the microwave signals received from each of the electrodes. At step 430, the multiplexed signal can be provided to a radiometer, which, as represented by step 435, can compare an internal reference temperature with the temperature information from the multiplexed signal to produce an output signal at the radiometer. At step 440, a controller may sample the output signal at the radiometer at a sampling rate to obtain the temperatures at the different locations where the electrodes or other energy delivery members are positioned.

Since radiometry technology is utilized, the temperature of targeted tissue at depth relative to the electrodes can be advantageously obtained. In some embodiments, such tissue temperature data can be used to regulate (e.g., either manually or automatically) the delivery of energy provided by each electrode or other energy delivery member to the adjacent tissue of the subject. For example, a physician or other practitioner can utilize the provided temperatures to increase the precision of a cardiac ablation procedure or other heat treatment procedure at the plurality of locations associated with the plurality of RF electrodes. In some arrangements, at step 445, the procedure ends.

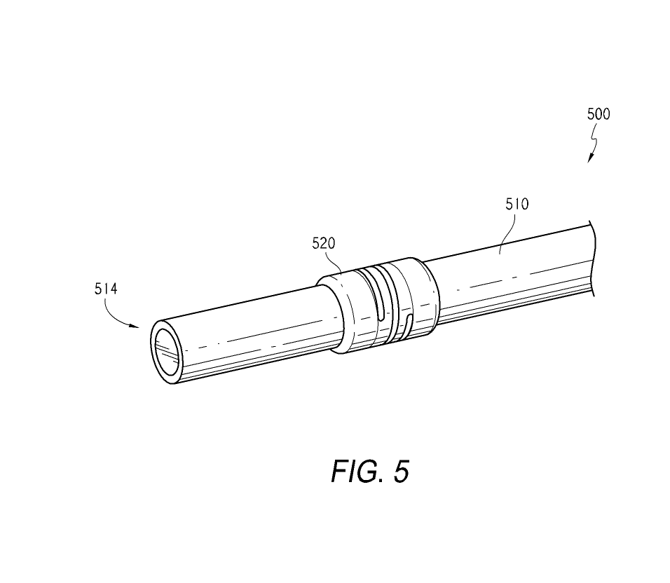

One embodiment of a RF electrode 520 that can be positioned along a catheter in a multi-electrode system 500, in accordance with the present application, is illustrated in FIG. 5. As shown, the electrode 520 can comprise a ring electrode that includes a generally cylindrical shape and can be sized, shaped and/or otherwise configured for placement along the outside of a catheter or other elongate body 510. In some embodiments, as illustrated in FIG. 5, the electrode includes an antenna (e.g., helical antenna) and/or any other feature or component that can facilitate receiving microwave signals. As discussed, such signals can be used to radiometrically determine the temperature of tissue at depth. In some embodiments, as discussed in greater detail below, one or more of the electrodes 520 can be positioned away from the distal end 514 of the catheter 510, depending on the desired location of the electrodes, the intra-electrode spacing and/or any other considerations.

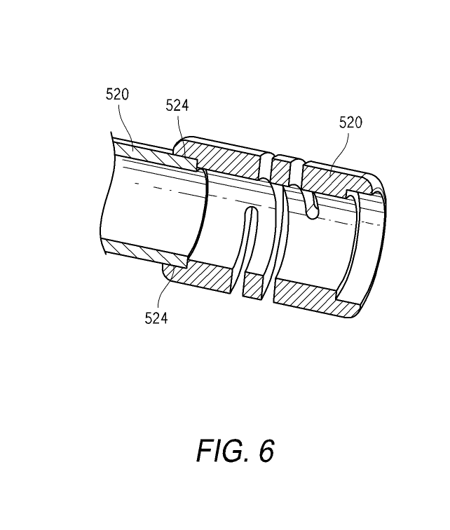

FIG. 6 illustrates a partial cross-sectional view of an electrode 520 that can be incorporated into a multi-electrode system 500. As shown, the proximal and/or the distal ends of the electrode 520 can be shaped, sized and/or otherwise configured to at least partially receive a portion of a catheter or other elongate member therein. For example, in the depicted arrangement, an end of the electrode 520 can include a counter-bore or other recess 524 that is configured to receive the catheter 510. In some embodiments, the outer diameter of the catheter 510 is identical or nearly identical (e.g., slightly larger or smaller) than the inside diameter of the counter-bore, recess or other feature 524 located at or near the adjacent end of the electrode 520. The catheter or other elongate member 520 can be secured to the electrode 520 using one or more attachment devices or methods, such as, for example, a press-fit connection, friction fit connection, glues or other adhesives, tabs, other mechanical fasteners and/or the like. Although not illustrated in FIG. 6, another section of catheter or elongate member can be positioned within the counter-bore along the other end of the electrode 520. Thus, in some embodiments, sections of catheter 510 can be used to connect the various electrodes 520 of the system 500 to one another in this manner.

In other embodiments, the catheter or other elongate member continuously extends through and/or within the various electrodes that are included in the multi-electrode system. For example, the plurality of electrodes can be positioned over a catheter or elongate body (e.g., secured to one or more exterior surfaces of a catheter or other elongate member). Regardless of the exact configuration of the catheter and the electrodes, one or more conductors can be used to couple the various electrodes to an energy delivery module (e.g., a RF generator), to couple the antennas to radiometer components (e.g., integrated chips) and/or to couple other components of the system to one another, as desired or required.

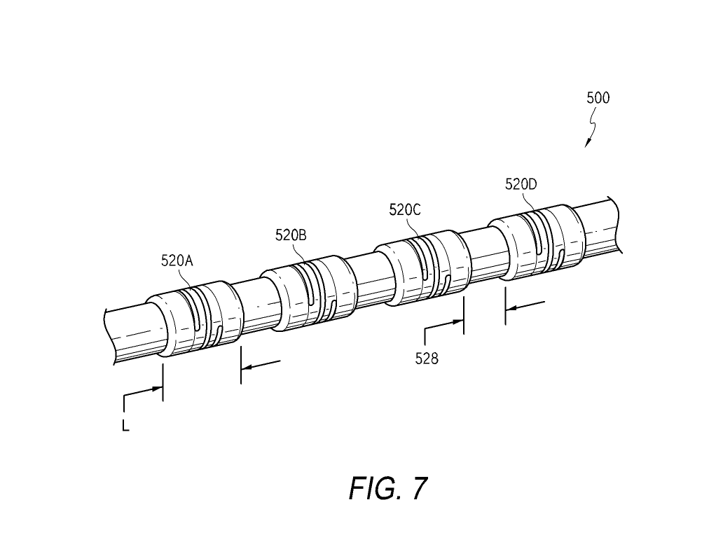

One embodiment of a system 500 can includes a plurality of electrodes 520A-520D is illustrated in FIG. 7. As shown, the system can include a total of four electrodes 520A, 520B, 520C, 520D. However, in other embodiments, the number of electrodes included in a system can vary (e.g., 2, 3, 5, 6, 7, 8, 9, 10, 10-15, more than 15, etc.). Regardless of the quantity of electrodes that are included in a system 500, the shape, size and/or other characteristics of each electrode can be identical or similar. For example, the length, diameter, power output, spacing between adjacent electrodes and/or the like can be the same for all electrodes. In other embodiments, however, one or more of the electrodes can include a different design or configuration than other electrodes. For example, the length or diameter of one or more electrodes included in a system can vary. In some arrangements, some electrodes are configured to deliver more or less energy to adjacent tissue than other electrodes when energized. In other configurations, the outer shape of two or more electrodes can vary, as desired or required. For example, one electrode comprises a circular outer shape, whereas another electrode comprises an oval, polygonal (e.g., hexagonal, octagonal, etc.), irregular or other non-circular outer shape. Accordingly, in some embodiments, the electrodes and their configuration (e.g., length, output, spacing, orientation, etc.) are selected based on the desired heating profile to tissue once the system is activated.

With continued reference to FIG. 7, the electrodes 520A-520D can be spaced apart from each other by a separation distance 528. For example, in some embodiments, the separation distance 528 (e.g., the distance from a distal end of a proximal electrode 520C to a proximal end of a distal electrode 520B) is between 1 mm and 10 mm (e.g., 1-2, 2-3, 3-4, 4-5, 5-6, 6-7, 7-8, 8-9, 9-10 mm, distances between the foregoing, etc.). In other embodiments, the separation distance is less than 1 mm (e.g., 0.1-0.2, 0.2-0.3, 0.3-0.4, 0.4-0.5, 0.5-0.6, 0.6-0.7, 0.7-0.8, 0.9-1 mm, values between the foregoing ranges, etc.) or greater than 10 mm (e.g., 10-50, 50-100, 100-500 mm, greater than 500 mm, values between the foregoing ranges, etc.), as desired or required. Although the separation distance between adjacent electrodes is illustrated in discussed with reference to FIG. 7, the foregoing disclosure can apply to any embodiments discussed herein or equivalents thereof. The separation distance 528 between each electrode may be uniform or non-uniform.

Moreover, the length L of an electrode 520A-520D incorporated into a multi-electrode system 500 can vary based on the particular application or use. For example, the length of an electrode can be between 2 mm and 8 mm (e.g., 2, 3, 4, 5, 6, 7, 8 mm, lengths between the foregoing values, etc.). In some embodiments, the length L of an electrode can be less than 2 mm (e.g., 0.01-0.05, 0.05-0.1, 0.1-0.2, 0.2-0.3, 0.3-0.4, 0.4-0.5, 0.5-0.6, 0.6-0.7, 0.7-0.8, 0.8-0.9, 0.9-1 mm, lengths between the foregoing values, etc.) or greater than 8 mm (e.g., 8-9, 9-10, 10-11, 11-12, 12-13, 13-14, 14-15, 15-20 mm, lengths between the foregoing ranges, greater than 20 mm, etc.), as desired or required. As noted herein, the shape, size and/or other properties of the various electrodes can be identical to one another or vary, depending on the particular design or application. Likewise, the diameter of the catheter or other elongate member 510 of the system 500 can vary. For example, in some embodiments, the catheter 510 can comprise a 3 French to 15 French (e.g., 3, 4, 5, 6, 7, 8, 9, 10, 11, 12, 13, 14 French, sizes between the foregoing, etc.) catheter. The outer diameter of the catheter 510 can be 1 to 3 mm (e.g., 1-2, 2-3 mm, diameters between the foregoing ranges, etc.). In some embodiments, however, the outer diameter of the catheter 510 is less than 1 mm (e.g., 0.01-0.1, 0.1-0.5, 0.5-1 mm, diameters between the foregoing, etc.) or greater than 3 mm (e.g., 3-4, 4-5, 5-6, 6-7, 7-8, 8-9, 9-10, 10-15 mm, diameters between the foregoing ranges, greater than 15 mm, etc.).

As noted herein, according to some embodiments, the plurality of electrodes included on a catheter of a system can comprise ring electrodes (e.g., electrodes having a generally cylindrical or ring shape) that extend circumferentially around the catheter. However, in other embodiments, the electrodes can comprise any other shape. For example, one or more of the electrodes included in a system can comprise non-ring electrodes and/or can extend only partially around the catheter or other elongate member. In some embodiments, one or more of the electrodes extend 0-45, 0-90, 0-135, 0-180, 0-225, 0-270, 0-315, 0-360 degrees around the catheter, angles between the foregoing range, etc. Further, in some embodiments where electrodes do not extend circumferentially around the entire catheter, two or more of the electrodes can be radially offset from one another. Thus, the different radial portions of the adjacent tissue (e.g., wall of a vessel or other lumen) can be targeted.

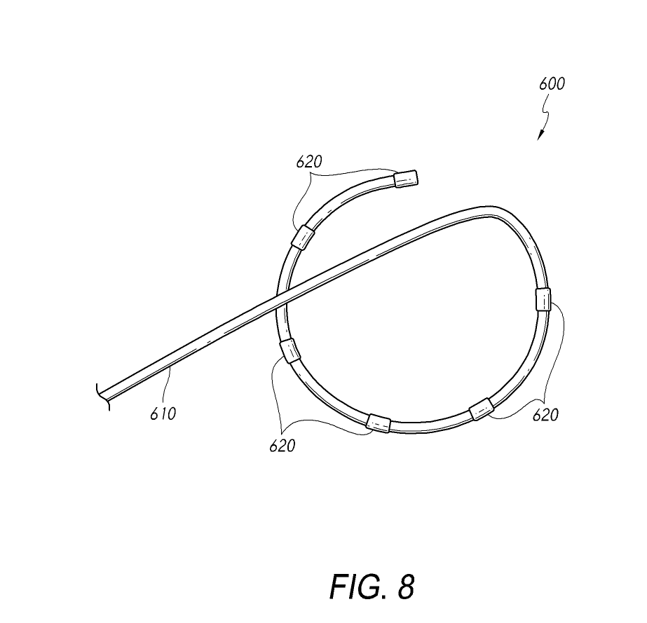

According to some embodiments, one or more of the electrodes can be positioned along a structure or member. In some embodiments, the structure or member on which one or more electrodes are positioned can be expandable or otherwise moveable between two or more positions. However, in other embodiments, as discussed herein with reference to FIG. 8, the structure that supports the electrodes can be fixed or non-movable.

In some embodiments, an expandable structure or member can include an expandable balloon (e.g., an inflatable balloon which can be compliant or non-compliant), an expandable cage, scaffold or other structure and/or the like. In some embodiments, the expandable cage comprises two or more fingers, struts, prongs and/or other members that can be selectively moved between a radially contracted position and a radially expanded position. In some arrangements, one or more of the electrodes positioned along an expandable balloon or other structure are configured to contact targeted tissue when such a balloon or other structure is radially expanded. In other embodiments, however, depending on the type of energy delivery members that are positioned along a catheter or other elongate member of the system, the energy delivery members need not contact tissue to deliver the necessary ablative or other energy to said tissue during a treatment procedure. In embodiments that comprise an expandable cage or other expandable structure, one or more electrodes or other energy delivery members can be positioned along each of the struts, fingers, prongs or other members of the cage or other structure.

In some embodiments, as illustrated in FIG. 8, for example, electrodes 620 can be positioned radially or circumferentially along one or more fixed structures. For example, in the depicted arrangement, the electrodes are positioned in a radial manner (e.g., generally perpendicular to the axis of catheter 610) along a circular or curved structure located at or near the distal end of the catheter 610. Accordingly, such a configuration can be used to simultaneously contact and treat various portions of non-linear (e.g., curved) tissue, such as, for example, vessels or other bodily lumens, curved or other non-linear portions of an organ and/or the like. Further, in other embodiments, electrodes can be positioned along other structures, including, but not limited to fixed or movable cages, struts, fingers, prongs or other members.

In some embodiments, the various systems disclosed herein can be configured to confirm contact between the electrodes and adjacent target tissue. For example, in some embodiments, the system can be configured to confirm tissue contact between an electrode and the targeted tissue before that specific electrode can be activated to supply ablative energy to such targeted tissue. In embodiments, where two or more electrodes will be activated simultaneously during a procedure, the system can be configured to ensure that all electrodes that will be activated are in contact with such tissue before ablative energy is supplied by such electrodes to targeted tissue. Additional details regarding confirmation of tissue contact are provided in U.S. application Ser. No. 12/483,407, filed on Jun. 12, 2009 and issued as U.S. Pat. No. 8,206,380 on Jun. 26, 2012, and U.S. application Ser. No. 13/486,889, filed on Jun. 12, 2012 and published as U.S. Publ. No. 2013/0324993 on Dec. 5, 2013, the entireties of both of which are hereby incorporated by reference.

The foregoing description describes only certain embodiments. However, other variations and modifications may be made to the described embodiments, with the attainment of some or all of their advantages. For instance, it is contemplated that although the description of the illustrated embodiments refer to RF electrodes as the energy delivery members, the concepts disclosed herein may be applied to a variety of different types of ablation catheters, and specifically to those that employ microwave antennas (e.g., utilizing microwave energy), ultrasound transducer, devices associated with cryoablation and/or any other devices that may be utilized to perform the ablation processor at the locations.

Further, although reference is made to RF electrodes that are configured to perform both ablation and temperature sensing, the systems can utilize one or more RF electrodes to perform the ablation or other heat treatment procedure within a zone of targeted tissue, while one or more other RF electrodes perform the temperature sensing at different locations, or vice versa.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

While the embodiments disclosed herein are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “advancing a catheter” or “delivering energy to an ablation member” include “instructing advancing a catheter” or “instructing delivering energy to an ablation member,” respectively. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 mm” includes “10 mm.” Terms or phrases preceded by a term such as “substantially” include the recited term or phrase. For example, “substantially parallel” includes “parallel.”

1. An ablation system comprising:
an elongate body comprising a proximal end and a distal end;
a first energy delivery member positioned along the distal end of the elongate body;
at least a second energy delivery member positioned at a location proximal to the first energy delivery member, the first energy delivery member and the second energy delivery member being configured to deliver energy sufficient to at least partially ablate tissue;
wherein each of the first and second energy delivery members comprises an antenna configured to receive a microwave signal corresponding to a temperature of the tissue at a location adjacent the antenna;
at least one radiometer configured to process the microwave signals received from the antennas of the first and second energy delivery members, the at least one radiometer being configured to produce an output signal representative of tissue temperatures at depth adjacent the first and second energy delivery members;
an energy delivery module; and
at least one conductor coupling the first and second energy delivery members to the energy delivery module.

2. The system of claim 1, wherein each of the first and second energy delivery members comprises a radiofrequency (RF) electrode.

3. The system of claim 1, wherein each of the first and second energy delivery members comprises at least one of a microwave emitter, an ultrasound transducer an optical emitter and a cryoablation member.

4. The system of claim 1, wherein the first and second energy delivery members are positioned axially along the elongate body.

5. The system of claim 1, wherein the first and second energy delivery members are positioned radially relative to the elongate body.

6. The system of claim 1, wherein each of the antennas of the first and second energy delivery members comprises a helical antenna.

7. The system of claim 1, wherein each of the first and second energy delivery members extends circumferentially around the elongate body.

8. The system of claim 1, wherein each of the first and second energy delivery members does not extend circumferentially around the elongate body.

9. The system of claim 8, wherein the first energy delivery member is radially offset from the second energy delivery member.

10. The system of claim 1, wherein the first and second energy delivery members are positioned on an expandable member.

11. (canceled)

12. (canceled)

13. (canceled)

14. The system of claim 1, wherein the elongate body comprises at least one irrigation passage, said at least one irrigation passage extending to at least one of the first and second energy delivery members.

15. The system of claim 14, wherein the irrigation passage is part of an open irrigation system, wherein fluid delivered through the at least one irrigation passage exits the elongate body near at least one of the first energy delivery member and the second energy delivery member.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. An ablation system comprising:
a plurality of energy delivery members configured to deliver energy sufficient to at least partially ablate the tissue when activated;
wherein each of the plurality of energy delivery members comprises an antenna configured to receive a microwave signal corresponding to a temperature of the tissue at a location adjacent the antenna;
at least one radiometer configured to process the microwave signals received from the antennas of the energy delivery members, the at least one radiometer being configured to produce an output signal representative of tissue temperatures at depth adjacent the energy delivery members; and
at least one conductor coupling the energy delivery members to an energy delivery module.

30. The system of claim 29, wherein each of the energy delivery members comprises a radiofrequency (RF) electrode.

31. The system of claim 29, wherein each of the energy delivery members comprises at least one of a microwave emitter, an ultrasound transducer, an optical emitter and a cryoablation member.

32. The system of claim 29, wherein the energy delivery members are positioned axially and/or radially along an elongate body.

33. The system of claim 29, wherein each of the antennas of the energy delivery members comprises a helical antenna.

34. The system of claim 29, wherein each of the energy delivery members is positioned on an elongate body, wherein each of the energy delivery members extends circumferentially around said elongate body.

35. The system of claim 29, wherein each of the energy delivery members is positioned on an elongate body, wherein each of the energy delivery members does not extend circumferentially around said elongate body.

36. The system of claim 35, wherein a first energy delivery member is radially offset from a second energy delivery member.

37-70. (canceled)
